(19) United States
(12) Patent Application Publication
(10) Pub. No.: US 2015/0201856 A1
(43) Pub. Date: Jul. 23, 2015

(54) ELECTRODE AND MEASURING DEVICE FOR ACQUIRING BIOMEDICAL VITAL PARAMETERS

(71) Applicant: KARLSRUHER INSTITUT FUER TECHNOLOGIE, KARLSRUHE (DE)

(72) Inventors: Wilhelm Stork, Landau (DE); Silvester Fuhrhop, Karlsruhe (DE); Stefan Lamparth, Karlsruhe (DE); Malte Kirst, Karlsruhe (DE)

(21) Appl. No.: 14/418,078

(22) PCT Filed: Jul. 25, 2013
(86) PCT No.: PCT/EP2013/002204

§ 371 Date: Jan. 29, 2015

(30) Foreign Application Priority Data
Jul. 30, 2012 (DE) 10 2012 106 893.3

(51) Int. Cl. A61B 5/0408; A61B 5/11; A61B 5/021; A61B 5/053; A61B 5/00; A61B 5/01

(52) U.S. Cl. CPC A61B 5/0408; A61B 5/6804; A61B 5/01; A61B 5/02125; A61B 5/0533; A61B 5/0535; A61B 5/1118

(57) ABSTRACT

A skin-contact electrode for registering a biomedical vital parameter includes a component assemblage comprising an outer edge, a first component and a second component. The first component has a planer design and comprises an electrically conductive material having a first component rigidity. The second component comprises an electrically insulating material having a second rigidity. The first component rigidity is greater than the second component rigidity. The outer edge is only formed by the second component.

### CROSS REFERENCE TO PRIOR APPLICATIONS

This application is a U.S. National Phase application under 35 U.S.C. §371 of International Application No. PCT/EP2013/002204, filed on Jul. 25, 2013 and which claims benefit to German Patent Application No. 10 2012 106 893.3, filed on Jul. 30, 2012. The International Application was published in German on Feb. 6, 2014 as WO 2014/019662 A1 under PCT Article 21(2).

### FIELD

The present invention relates to a skin-contact electrode for registering biomedical vital parameters. It comprises a first component with a planar design, which is made of an electrically conductive material, as well as a measuring device for registering the vital parameters and a carrying device made of an elastic material, which is detachably connected to a data processing unit. It includes at least one skin-contact electrode electrically connectable to the data processing unit.

### BACKGROUND

For the purposes of registering, analyzing, transmitting and reporting on vital parameters of a user, measuring devices with electrodes, which are integrated into a carrying device and designed to register the heartbeat of a user, have previously been described. vital parameters are here measures which reflect the basic functions of the human body; by way of example, these include the heart rate, the blood pressure, the body temperature and the respiratory rate. These furthermore include the electrocardiogram (ECG) and electroencephalography (EEG). In addition to the electrodes as sensors, known types of carrying devices include at least one recording device, which is signal connected to the sensors, for registering the parameters.

The carrying devices for long durations of application known from the prior art are embodied as chest straps or integrated into pieces of clothing. Such a carrying device is described in DE 10 2005 004 443 A1, which integrates sensors into a piece of clothing, in this case a T-shirt.

The prior art has disclosed different types of electrodes, wherein these often consist of conductive, flexible films. These conductive planar materials serve firstly as an electrode and secondly as electrical supply lines for a connection to the processing electronics, as is also described in DE 11 2004 001 921 T5, which describes a sensor arrangement for measuring signals on the surface of the skin. The electrode surfaces or the electrical supply lines, which are required for transmitting a signal, are fastened by being coated onto a textile material of a piece of clothing or of an accessory, and are to be worn directly on the skin. DE 10 2009 052 615 A1 describes planar materials for an electrical connection. DE 10 2009 052 615 A1 also describes a measuring recorder for ECG signals comprising a transmission unit and a chest strap made of an elastic material, wherein at least three electrodes are applied to the chest strap, which electrodes are electrically connected to measurement electronics by means of a planar conductor. Use is likewise made of films for insulating the electrical supply line from the body of the user. The area covered thereby is overall not vapor-permeable, which substantially reduces the comfort of wear, particularly in the case of long-term applications.

The electrically conductive conduction structures in the prior art were until now contacted by means of snap fasteners. The number of electrical contacts and the number of supply lines contactable thereby are restricted due to the size thereof. The planar conductors moreover have a certain minimum width, which furthermore puts restrictions on the supply lines placed in a textile, especially those in a chest strap. Due to manufacturing tolerances, the introduction of more than two conductive tracks running in parallel next to one another was difficult employing the technology (planar conductors) used up until now, as a result of which the number of electrodes or electrical connections in the textile are also restricted.

The use of films greatly restricts the elasticity of the carrying device in the region of the planar supply line. This should be noted, in particular, if, as is usually the case in the prior art, use is made of more than two electrodes.

In addition to electrodes made out of a conductive film, Ag/AgCl electrodes are used for deriving medical vital data. As is also described in DE 693 00 625 T2, these electrodes consist of a substrate made of Ag/AgCl which is adhesively bonded onto the skin of the user by means of a self-adhesive edge. This type of electrode is not very durable and cannot be reused after one application. Such electrodes also have the disadvantage that skin macerations and skin irritations connected therewith arise in the case of a time of wear of more than 24 hours.

In the prior art, measuring devices for recording electrophysiological signals are equipped with flexible dry electrodes. These are, for example, described in CA 2 620 578 A1. These are usually fixed on the body of the user by means of a flexible, textile carrying device. As a result of the flexibility of the electrodes, the textile on the body of the user has a uniform contact pressure everywhere. In order to achieve a high signal quality, it is, however, necessary for the textile to press the electrodes against the body with a high contact pressure. This is uncomfortable in the long run and justifies the desire for a lower contact pressure, particularly from ergonomic points of view.

In addition to a low comfort of wear, a disadvantage of the aforementioned electrodes in the case of a long-term application lies in the low signal quality of these electrodes since signal interference in the form of movement artifacts is caused by moving skin sections when the surface signals are measured.

Movement artifacts have for a long time been a problem for measurements within the scope of long-term electrocardiogram (ECG) monitoring of diseased patients and within the scope of an ECG stress test and are generated at the interface between electrode and body surface, wherein said movement artifacts reduce the quality of the derived signal and make the signal processing or additional processing more difficult. The reasons therefor exist mainly in the relative movements between the electrode and the body surface, and in skin potentials generated by the changes in pressure on the body surface. The interfering potentials are greater in the edge region than in the center of the electrically conductive electrode surface since shearing forces occur increasingly in the edge region. A movement generates an external output on the monitor, which either masks the desired biopotential or shifts the baseline, as a result of which the usefulness of the electrode, the diagnosis or the clinical instrument per se is reduced.

The intensity of movement artifacts is approximately inversely proportional to moisture, i.e., the electrolyte between the electrode surface and the body surface, so that these artifacts occur especially in the case of dry electrodes. The moisture between the electrode and skin for establishing an electrical contact in these is achieved by the formation of sweat. However, moisture evaporates more in the edge region of the electrode than in the center, which is why there is less moisture prevalent there. The edge effects present, i.e., the movement artifacts, are therefore amplified further.

For reducing the movement artifacts in the electronics and for improving the comfort of wear, it is therefore expedient to increase the contact pressure at the electrode and to minimize the contact pressure of the carrying textile, required for this, in all regions.

### SUMMARY

An aspect of the present invention is to provide an electrode which makes it possible to record electrophysiological surface signals with a high signal quality and which enables a long duration of application. A further aspect of the present invention is to provide a measuring device with good ergonomics which is producible in a simple and cost-effective manner.

In an embodiment, the present invention provides a skin-contact electrode for registering a biomedical vital parameter which includes a component assemblage comprising an outer edge, a first component and a second component. The first component has a planer design and comprises an electrically conductive material having a first component rigidity. The second component comprises an electrically insulating material having a second rigidity. The first component rigidity is greater than the second component rigidity. The outer edge is only formed by the second component.

### BRIEF DESCRIPTION OF THE DRAWINGS

The present invention is described in greater detail below on the basis of embodiments and of the drawings in which:

FIG. 1 shows a schematic view of a measuring device according to the present invention;

FIG. 2 shows a plan view of an embodiment of an electrode according to the present invention;

FIG. 3 shows a plan view of an embodiment of the electrode according to the present invention;

FIG. 4 show a cross-section through an embodiment of the electrode according to the present invention;

FIG. 5 show a cross-section through an embodiment of the electrode according to the present invention;

FIG. 6 show a cross-section through an embodiment of the electrode according to the present invention;

FIG. 7 show a cross-section through an embodiment of the electrode according to the present invention;

FIG. 8 show a cross-section through an embodiment of a fastening of the electrode according to the present invention;

FIG. 9 show a cross-section through an embodiment of a fastening of the electrode according to the present invention;

FIG. 10 show a cross-section through an embodiment of a fastening of the electrode according to the present invention;

FIG. 11 show a cross-section through an embodiment of a fastening of the electrode according to the present invention;

FIG. 12 shows a cross-section through a carrying device comprising an electrode according to the present invention, resting on skin of a user;

FIG. 13 shows a cross-section through a carrying device comprising an electrode according to the present invention, resting on skin of a user;

FIG. 14 shows a perspective side view of the measuring device according to the present invention;

FIG. 15 shows a schematic side view of the measuring device according to the present invention;

FIG. 16 shows a schematic side view of the measuring device according to the present invention; and

FIG. 17 shows a schematic side view of the measuring device according to the present invention.

### DETAILED DESCRIPTION

The present invention relates to a skin-contact electrode for registering biomedical vital parameters, including a first component with a planar design, which is made of an electrically conductive material. Here, the first component abuts on a second component, which is made of an electrically insulating material and forms a component assemblage with the first component. The first component has a more rigid embodiment compared to the second component, wherein an outer edge of the component assemblage is only formed by the second component.

The first component of the electrode can, for example, have a rigid embodiment and be surrounded by a soft second component. “Rigid” in this case means a certain stability of the first component relative to the second component and relative to the carrying device itself. The present invention can, for example, provide for the electrode to be fastenable onto a carrier device, wherein the component assemblage of the electrode has a more rigid embodiment compared to the carrier device.

The first component can be present in various embodiments, wherein it can have an integral or multi-part embodiment. What is common to all embodiments is, however, that they are embodied in a manner more rigid in relation to the second component and do not record any movement of the applied skin surface as such. The first component of the electrode may, for example, include a metal, in particular stainless steel or titanium. This embodiment is friendly to the skin and suitable for long-term applications. Use can also be made of other metallic surfaces with a smooth or porous structure, in particular a conductive textile which, as a fiber composite, contains conductive woven fabrics, loop-formingly knitted fabrics or loop-drawingly knitted fabrics.

In an embodiment of the present invention, provision can, for example, be made for the first component to include a flexible, conductive film or a conductive textile and for the film or the textile to be connected at a contact surface to a substrate, which is comparatively rigid compared to the film, and thus form the first component. By way of example, suitable materials are thermoplastic polyurethane (TPU), to which carbon or charcoal was added as a conductive medium, or thermoplastic elastomers (TPE) in the form of sheets or films and further thermally deformable plastics in the form of sheets or films. Use can also be made of vulcanizable plastics.

In an embodiment of the present invention, provision can furthermore be made, for example, for the first component to be made up of two film plies, wherein a conductive textile, which is electrically connected to an electrical supply line, is pressed or adhesively bonded between the two plies. The adhering can also be brought about by thermoplastic methods, as a result of which the conductive woven fabric is rather fused between the thermoplastics. The second component here contacts the first component over the whole area thereof. In order to achieve an electrode which is rigid overall, the second component can furthermore be arranged in a manner contacting the substrate over the whole area thereof. The conductive textile can be present as a silver-plated, low-resistance conductor. The embodiment of the electrode is comfortable to wear and producible in a cost-effective manner. Any hardness/degree of hardness of the electrode desired can be set by various strengths of the second component in order to thereby achieve the optimum between ergonomics and signal quality.

In an embodiment of the present invention, provision can, for example, be made for the second component to have a coefficient of static friction for adhesion of the component assemblage to biomedical skin. The second component advantageously includes a flexible material, for example, silicone. The second component is therefore embodied as an adhesive edge which, although it is suitable for adherence, does not stick. This enables good contact of the electrode on a skin portion of the user, wherein the electrically active electrode surface of the first component is always in complete contact with the body in the case of relative movement between the body and electrode. As a result of the electrical insulation of the adhesive edge, signal interferences in the region of the edge, such as e.g., shear loads and movement artifacts of the electrophysiological signal conducted away by the electrically active electrode surface of the first component, are prevented.

In an embodiment of the present invention, the first and second components can, for example, include skin-contact surfaces which are embodied as lying in a plane. The first and second component therefore rest on the skin of the user in a planar manner by way of their contact surface. The skin-contact surface of the first component can also be embodied in a manner raised in relation to the second component, wherein an outer edge of the component assemblage consisting of the first and second component may be formed by the second component and the first component can be increased in height relative to the second component. A whole-area contact of the first component and an adhesion of the second component on the skin of the user are in any case provided.

For the purposes of good contact between the electrode and the skin portion of the user, the surface of the first component can have a planar embodiment or elevations embodied in a regular or irregular manner. There can be good contact with the skin portion as a result, even in the case of much hair or other skin unevenness.

In order to achieve protection of the first component against kinking, provision can, for example, be made for the second component to wholly or partly engage around an edge portion of the first component. A laterally formed limb can also be enclosed by the second component. The first component is therefore not only protected against kinking, but also electrically insulated along the edge. For a comfortable wearing sensation and as a protection against kinking, the edge of the second component can be embodied in a sloping manner on a side facing away from the skin-contact surface, wherein the second component can have a thinner embodiment toward the edge and therefore become more flexible. This form of the second component can be generated by simple fusion with the carrier device, wherein the material of the second component partly sinks into the material of the carrier device and completely surrounds the edge of the first component.

The electrical contacting is brought about by means of soldering, welding or (thermal) adhesion at the first component, for example, on the lower side thereof, by means of a cable, wherein solder tails or solder lugs or other attachment means can be formed on the first component for this purpose. Other individually insulated electrical supply lines are also suitable.

If electrically conductive thermoplastic films are used as the active electrode surface, the supply line can also be mechanically and electrically contacted by inserting and thermally pressing the stripped end thereof between the thermoplastic films.

In an embodiment, the present invention provides a measuring device for registering biomedical vital parameters which comprises a carrying device made of an elastic material, which can, for example, be detachably connected to a data processing unit and which includes at least one skin-contact electrode electrically connectable to the data processing unit.

As described above, the electrode can be present in various embodiments. As a result of the comparatively rigid first component of the component assemblage, the electrode is substantially more rigid than the underlying carrying device, which usually consists of an elastic material. In the skin-contact area, the body surface of a user is always convex, as a result of which the electrode deforms the relatively soft body surfaces in a specific region of the contact surface thereof. As a result of the rigid electrode, the carrying device is moreover lifted away from the body in the edge region of the electrode, as a result of which the contact pressure in the region of the active electrode surface, i.e., the first component, is increased.

The present invention can furthermore provide for the contact surface of the skin-contact electrode to be fastened to the carrying device in a raised manner so that the skin-contact surface of the first component is raised in relation to a surface of the carrying device. It can likewise have a planar design on the side of the carrying device facing the body. After installation, the electrically active surface does not then lie under the surface of the carrying device, as a result of which the latter can be worn with less contact pressure overall, resulting in a significant improvement in the comfort of wear.

Provision is in particular made for two, three or more skin-contact electrodes to be attachable to the carrying device, wherein at least one skin-contact electrode is arranged on a portion of the carrying device that can be placed on the back of the user. Four electrodes are necessary, especially for exactly recording an ECG. In the case of more electrodes, the remaining electrodes can find use in measuring other vital parameters. It is also possible for the electrodes employed for the ECG to be used in a twofold manner for registering other vital data. Depending on the position and arrangement of the electrodes, this allows particularly robust and exact measurements of the vital parameters to be performed as a result of the redundancy in the electrodes.

In addition to a skin-contact electrode for registering vital parameters, which is provided as a minimum, further sensors can be provided which, in an embodiment, can be acceleration sensors which are in particular arranged at or within the carrying device. The measuring device can thereby be used for many different measurement applications for vital parameters of a user.

The measuring device should be able to record vital parameters of a user, wherein at least the ECG can be derived. As a result of using a plurality of electrodes, redundant signal channels are obtained, as a result of which the signal quality can be further improved. The measuring device can in particular undertake the following measurements: an impedance of the body or a body part, such as the thorax, can be measured by means of at least two electrodes or resistive, inductive or capacitive structures. By means of an activity sensor, in particular an acceleration sensor, it is possible to record the physical activity and the current body position. A development of an embodiment of the present invention includes a plethysmograph or a GSR sensor. In addition to ECG parameters, it is thus also possible to record the pulse-transit time or the blood pressure or blood pressure equivalent. Provision can also be made of a sensor that is suitable for measuring the temperature.

In order to fasten the electrode to a measuring device or a different surface, provision is made according to the present invention for the electrode to have at least one perforation serving for fastening the electrode at the edge. As a result, different connection means can be employed; the electrode can thus be connected to a measuring device circumferentially by sewing, crimping or adhering. A multiplicity of such perforations can, for example, be provided in the edge of the first component, wherein it is also possible for the second component to be embodied as extending over the edge portion of the first component and for the second component to be fused in these edge regions and by means of the perforations to a thermoplastic connection ply situated under the first component. Further fastening means, such as, for example, screws, rivets or the like, can furthermore be provided; these are arranged in the edge region of the first component or of the electrode.

The skin-contact electrode can be wholly or partly fused to the carrying device by means of the thermoplastic connection ply. A permanent, cohesive connection is generated thereby. Provision can also be made for the electrode to be fixed not onto, but in the interior of, the carrying device.

Dimensions of the electrodes can, for example, have a thickness of the electrodes of between 0.2 and 3 mm. However, thicker or thinner electrodes can also be realized with the present invention. In the planar extent, the electrodes may have the following dimensions in an exemplary manner: 2×2 cm, 3.5×4 cm, 4×6 cm, 5×7 cm, 7×9 cm. Other dimensions are, however, also possible.

The present invention provides for a body part of a user to be able to be engaged wholly or partly by the carrying device. To this end, the carrying device can be a chest strap, an armband, a foot band or a bracelet. The skin-contact electrodes are also integrable into pieces of clothing, in particular into stockings, gloves or T-shirts. The carrying device provides two free ends that can be connected by a closure, particularly in the case of an embodiment as a chest strap. The closure can have such an embodiment that it enables a length adjustment of the carrying device.

In an embodiment, the present invention can, for example, provide for the carrying device to be made from a textile made of a synthetic material. The textile can be present as a woven fabric, a loop-drawingly knitted fabric, or a loop-formingly knitted fabric, in particular as a web-like plastic. In addition to synthetic materials, use can also be made of natural materials such as cotton. As a result of the selection of material, the carrying device between the electrodes has a highly flexible embodiment, whereby the electrodes remain at one location and the carrying device absorbs the extension or compression coupled in by the body movement. Textile materials in particular are advantageously machine-washable and have a high vapor permeability.

In order to electrically and mechanically connect the electrodes or further sensors of the carrying device to the data processing unit, the carrying device includes electrical and mechanical connection means. At least one connection means can be embodied as a multi-pole plug, which is electrically connected to the at least one skin-contact electrode and possible further sensors by means of individually routed cables or a cable harness. It is possible as a result to increase the number of supply lines and, directly, the number of usable sensors and electrodes without running into the aforementioned disadvantages. Such a plug is space-saving, advantageously machine washable and bio-compatible, in particular bio-tolerable. It has polarity reversal protection and the insulation thereof has a defined clearance and creepage distance which, especially in the medical product range, meets the appropriate requirements for the standard. It can furthermore be thermally fixed or sewed into a slit within the carrying device and, in addition to electrical contacting, simultaneously serves for mechanically fixing the data processing unit.

The plug can be equipped with a mechanical connection means as a lock, by means of which an inadvertent detachment of the connection is prevented. A further connection to the carrying material is established by a connection apparatus, wherein this second mechanical connection provides an additional fixation of the data processing unit. This connection can be realized by means of snap fasteners or snap-on closures. In a further embodiment, provision can, for example, be made for the mechanical connection means to fasten the data processing unit to the carrying device at a side of the data processing unit lying opposite to the electrical connection means, wherein an electrical connection means may be arranged at the edge of the data processing unit on the other side. The mechanical connection means can also be arranged in a space-saving manner under the data processing unit. This connection means can be adhesively bonded, crimped or thermally fixed and can, for example, be embodied as a snap fastener. In an alternative embodiment, the aforementioned connection means can also be provided as a second, additional electrical connection means.

In an embodiment, provision can, for example, be made for the carrying device for holding the cabling to have a two-ply embodiment; the plug and individual electrical supply lines connected therewith can be kept loosely therein. This loose, tube-shaped connection reduces parasitic coupling into the electrode lines since individual conductors only move insubstantially in relation to one another and are routed with a small distance from one another. The length of the supply lines is advantageously determined in the case of great extension of the carrying device. In this manner, the carrying device remains elastic during use in the region of the electrodes.

As a result of the cabling by means of individual electrical conductors, a maximum vapor permeability is advantageously made possible in all regions, except for below the electrodes, since the individual electrical conductors merely have diameters of the order of millimeters. Defined insulation values, which are relevant for medical products, are furthermore possible. These insulation values can, for example, be between 60 V AC continuously and 1500 V AC peak values. Extensibility and vapor permeability of the carrying device are therefore only determined by the material used therefor. Regions of the electrical supply lines to the individual electrodes or the further sensor system have no influence on the extensibility or vapor permeability.

A further advantage of the individually routed electrical supply lines within the carrying device lies in the fact that a larger number of lines or, in that case also, electrodes and sensors become connectable. Redundant signal measurements can thus be recorded from a plurality of ECG leads and the signal quality can, overall, be further improved.

The data processing unit includes a memory device, at least one processor and an energy supply, wherein the data processing unit can, for example, be connected in a detachable manner to the carrying device by means of the plug. It is mobile as a result and can, where necessary, be removed from the carrying device so that the carrying device itself can be washed independently of the electronic components.

By way of example, energy is supplied by means of a battery, wherein the energy supply may likewise include a module for obtaining energy from the surroundings, such as e.g., a solar cell.

The data processing unit furthermore contains a telemetry device for communicating with the surroundings. The data processing unit can thus be connected to a network device, a conventional PC and the Internet. An amplifier circuit within the data processing unit provides conversion and preprocessing of the measured signals from the electrodes. Finally, the data processing unit is to be embodied to determine the GPS position of the user or of the measuring device. The data processing unit may include further sensors, e.g., acceleration sensors, for registering further parameters. A data memory medium and a user interface may also be provided. A small display can thus be attached, by means of which a user can independently set parameters such as measurement times of the measuring device by means of operating elements.

Advantages of the measuring device therefore include being particularly flexible and ergonomic, wherein the electrodes according to the present invention can be used to record electrophysiological surface signals, and therefore biomedical vital parameters, with a particularly high signal quality. Movement artifacts generated by body movements of the user are substantially reduced, if not even prevented altogether. The mechanical interference input as a result of the movement only changes the contact area of the electrically inactive edge, i.e., the second component, to a first approximation. The electrically active surface of the first component remains uninfluenced thereby. An ideal contact pressure is achieved, in particular, by the shape of the electrode in conjunction with the carrying device, said contact pressure being ideal in respect of the measurement and at the same time being wearable in an ergonomic and comfortable manner.

Additional embodiments, as well as some of the advantages connected thereto, become clearer and more easily understandable from the subsequent detailed description. The reference to the Figures in the description also assists in this context. Objects or parts thereof which are substantially the same or very similar may be provided with the same reference signs.

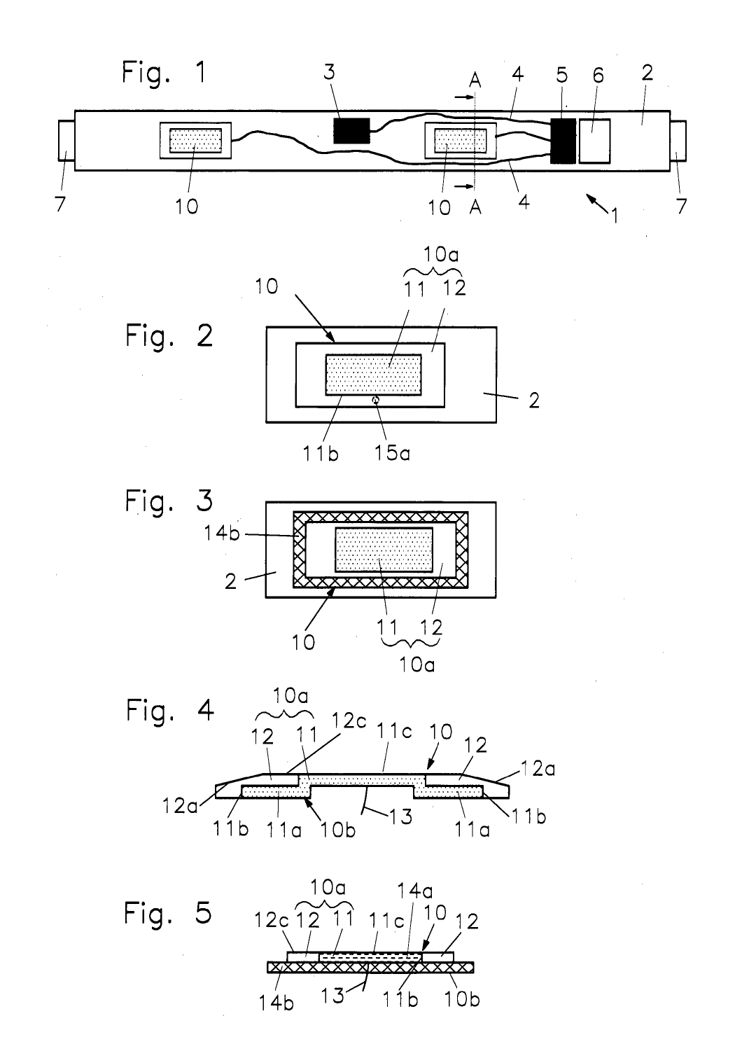

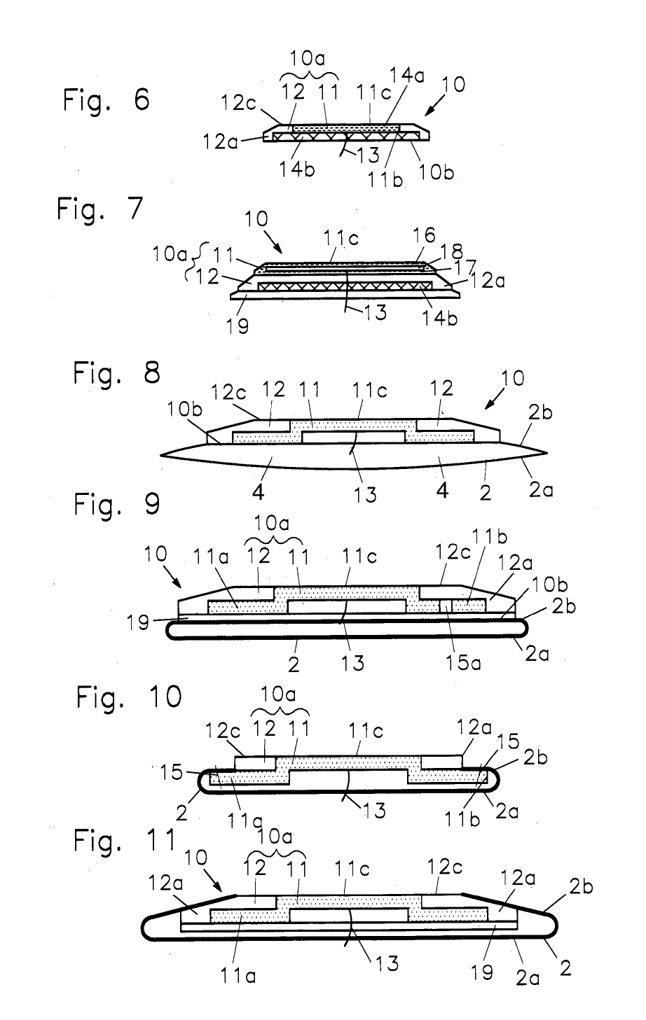

FIG. 1 depicts an embodiment of a measuring device 1 according to the present invention, which contains a carrying device 2 embodied as a chest strap, which is embodied as a chest strap with a rectangular elongate form. The carrying device 2 may consist of a textile or another synthetic material, which has a certain vapor permeability and which is at the same time elastic. The carrying device 2 provides two free ends that can be connected by a closure 7, particularly in the case of an embodiment as a chest strap. Two electrodes 10 as well as further sensors 3, e.g., a respiratory sensor, are arranged on the chest strap 2 in predetermined regions.

The robustness and accuracy of the measurement of the vital parameters increases as more electrodes 10 are provided. Advantageously, use can be made of three electrodes 10 for the purposes of keeping measurement errors and other interferences small. Two electrodes 10 should be applied in the chest region and one electrode 10 should be applied in the back region of the user.

The electrodes 10 and the sensor 3 are electrically connected to a plug 5 by way of cables 4. The cables 4 are present as insulated, individual electrical conductors, which are routed individually from associated contacts (e.g., plug pins, spring contacts) to the electrodes 10 or the sensor 3, wherein these are routed within the carrying device 2 which, to this end, has a two-ply embodiment in order to hold the cabling. They are not connected tightly to the respective ends so as to provide that the carrying device 2 remains elastic in the regions in which the cables 4 are routed and also to provide that a movement of the user does not damage the contact.

Assigned to the carrying device 2 is a data processing unit 6, which is detachably connectable to the plug 5 and serves as measurement electronics. For the data processing unit 6, the plug 5 serves not only as an electrical connection to the cabling 4, but also as a mechanical fixation, as a result of which it is integrated into the carrying device 2 by virtue of being sewn-in, adhesively bonded or thermally fixed. For the purposes of simple handling, the plug 5 has a pole reversal protection, i.e., the data processing unit 6 cannot be plugged-in in an inverted manner.

In addition to a measuring circuit in the form of an amplifier circuit, the data processing unit 6 contains a processor for signal processing, a memory device and a telemetry device for communication with the surroundings. The data processing unit 6 also includes a battery as a mobile energy supply. However, a module for obtaining energy from the surroundings, e.g., a solar cell, may also be installed.

In addition to the carrying device 2, the electrodes 10 can furthermore be connected to other devices and used as desired. In an embodiment, the electrodes 10 can also be applied individually to the skin of a user and electrically connected to a measuring unit by means of suitable connection means. This is made possible by the electrode 10 according to the present invention, which may be present in different embodiments.

FIGS. 2 and 3 show plan views of two selected embodiments of the electrodes 10 according to the present invention, which are substantially made from a component assemblage 10a with two planar-embodiment components arranged within one another, wherein, in FIG. 2, a first component 11 is completely surrounded by a second component 12 around the circumference thereof. The second component 12 forms an edge around the first component 11, the width of which edge varies depending on dimensions of the first component 11 arranged therein. The first component 11 is produced from an electrically conductive material, wherein both a metallic sheet and a conductive film are usable. The second component 12 consists of an electrically non-conducting material such as e.g., thermoplastic polyurethane (TPU), other plastics or silicone. Compared to the second component 12, the first component 11 is a more rigid embodiment, as a result of which the whole electrode 10 has a rigid embodiment, especially compared to the carrying device 2 situated therebelow.

At least one of the skin-contact surfaces 11c, 12c of the electrode 10 has a substantially rectangular form, wherein other geometric forms, such as a rectangle with rounded-off corners, an oval, a circle or a triangle, are also possible. The electrodes 10 are fastenable onto the carrying device 2 by virtue of an opening 15 for fastening the electrode 10 being provided in an edge portion 11b of the first component 11, as depicted in FIG. 2. By contrast, a thermoplastic substrate 14b is arranged under the electrode 10 in FIG. 3, which thermoplastic substrate 14b can be adhesively bonded or fused to the electrode 10 and the carrying device 2. The types of fastening will still be explained in more detail in the course of the following text.

In the subsequent FIGS. 4 to 7, different embodiments of the electrode 10 according to the present invention are depicted; these Figs. each show a cross-section according to section A-A according to FIG. 1.

What FIG. 4 depicts is that the first component 11 has an axisymmetric step-shaped embodiment in a section in relation to a central axis, wherein free limbs 11a define edge regions, on which the second component 12 is arranged. Furthermore, end portions 11b are surrounded by the second component 12, such that these are protected against damage and kinking.

Furthermore, an edge portion 12a of the second component 12 has an embodiment sloping away to the side; this achieves a continuous transition between the electrode and carrying device, as a result of which the electrode 10 can be placed onto the skin of a user in an ergonomic manner. The first component 11 in this embodiment consists of metal and is made from a thin sheet by deep-drawing. The limbs 11a in this case serve for fastening the second component 12, which can be securely and permanently adhesively bonded thereon.

The first component 11 and the second component 12 are arranged relative to one another in such a way that their skin-contact surfaces 11c, 12c lie in a plane; no gap is provided between the components 11, 12. The surface of the electrode 10 is therefore planar and can completely abut on the skin of a user.

On a lower side of the first component 11, the electrode 10 is connected to the measuring electronics by means of an electrical connection means, such as an electrical supply line 13, wherein the electrical supply line 13 is connectable to the first component 11 by means of soldering, welding or adhesively bonding. The electrode can be applied to a carrying device 2 via a contact surface 10b.

FIG. 5 depicts a further electrode 10, which corresponds to a sectional representation of the electrode 10 according to FIG. 3. The first component 11 is manufactured from a flexible, conductive electrode surface 14a, such as e.g., a conductive film, such as e.g., TPU-based films, to which carbon or charcoal was added as a conductive medium, or a conductive textile, such as e.g., woven fabrics, loop-drawingly knitted fabrics or loop-formingly knitted fabrics. The second component 12 is made of electrically insulating material and once again completely surrounds the first component 11 at the edge, whether this is in a view from above (FIG. 3) or from the side (FIG. 5).

In order to achieve rigidity of the first component 11, a rigid substrate 14b, onto which the electrode surface 14a and the second component 12 are adhesively bonded, is arranged on the lower side of the conductive electrode surface 14a. The electrode surface 14a and the substrate 14b form the first component 11 in this case. The substrate 14b may be embodied from a thermoplastic material, as a result of which the components 11, 12 can be fused or adhesively bonded to one another at their abutting surfaces and to the carrying device 2. The skin-contact surfaces 11c, 12c once again lie in a plane, as a result of which the first component 11 is surrounded by the second component 12 over the entire circumference thereof. Electrical contacting 13 is once again brought about on the electrode surface 14a.

FIG. 6 shows a development of the electrode 10 from FIG. 5, wherein the second component 12 is formed not only at the edge of the substrate 14b, but beyond the edges thereof and therefore laterally engages around the latter. In the section, the second component 12 is embodied in a partly sloping manner in the region of the edges of the substrate 14b. This is due to the fact that the second component 12 is thermally treated and loses height by temporary fusing in the edge portion 12a thereof. In a portion facing the user, the second component 12 is embodied in a plane with the first component 11, such that the contact surfaces 11c, 12c of the two components 11, 12 come to rest in a plane.

In an alternative electrode 10 in FIG. 7, the first component 11 is formed by a multi-ply setup, wherein an upper ply 16 and a lower ply 17 are connected to one another at the edge. The plies 16, 17 consist of conductive, flexible films. A conductive textile 18 is provided between the two plies 16, 17, which textile 18 is electrically connected to the electrical supply line 13 and pressed between the upper ply 16 and the lower ply 17. Once again, a substrate 14b is provided as a lowermost ply of the first component 11 in order to achieve the rigidity.

For the purposes of stabilizing and strengthening the first component 11, the second component 12 is arranged on a substrate 14b over the whole area. The plies 16, 17 and the textile 18 are adhesively bonded or fused hereon. In order to connect the electrode 11 manufactured thus to the carrying device 2 and in order to embody the electrode 10 in a more rigid manner, provision is furthermore made for a thermoplastic connection ply 19. The second component 12, in particular, extends over the whole area thereof.

FIGS. 8 to 11 depict various fastening variants of the electrode 10 according to the present invention on the carrying device 2.

In FIG. 8, the electrode 10 according to the present invention, as depicted in FIG. 4, is adhesively bonded to the surface of the carrying device 2. The carrying device 2 has a side 2a facing away from the body and a side 2b facing the body. The contact surface 10b here abuts on the carrying device 2 over the whole area of the body-facing side 2b. To this end, the lower-side regions of the electrode 10 are connected to the carrying device 2 by means of a permanent, water-insoluble and temperature-resistant connection, in particular by means of thermoplastic adhesion. The electrode 10 is therefore fastened to the carrying device 2 in a raised manner such that the skin-contact surface 11c of the first component 11 is raised in relation to the body-facing side 2b of the carrying device 2. After installation, the electrically active surface then does not lie under the surface of the body-facing side 2b of the carrying device 2, as a result of which the latter can, overall, be worn with less contact pressure.

Contacting of the first component 11 can take place in a preceding assembly step, wherein the electric supply line 13 can be routed through a small slit or other opening in the carrying device 2. The electric supply line 13 is connected to the cable 4 by means of soldering or crimping or has an integral embodiment with the cable 4, wherein the cable 4 is stripped in a certain portion at the free end to be fastened for establishing an electrical connection. To stop the electrical connection from cutting off as a result of extension of the carrying device 2, it can additionally be secured on the lower side of the first component 11. The carrying device 2 has a two-ply embodiment, wherein the electrical supply lines or cables 4 lie individually within the double ply and are thus protected from external influences and damage.

FIG. 9 depicts an electrode 10 embodied on the carrying device 2 in a raised manner, wherein the connection ply 19 is arranged below the first component 11. The first component 11 furthermore includes a multiplicity of perforations 15a, wherein only one perforation 15a is shown in FIG. 9. The second component 12 is applied by way of the perforation 15a. The edge region 11b is engaged laterally by the second component 12 and extends therebeyond to the extent that the second component 12 abuts with the lower side thereof on an upper side of the connection ply 19. Together with this portion, the perforation 15a is usable for interlocking fastening of the limb 11a to the connection ply 19 by virtue of the second component 12 being fusible with the thermoplastic connection ply 19 by means of a heat treatment at the overlap points thereof. The connection ply 19 can furthermore be partly or completely fused to the body-facing side 2a of the carrying device 2 in a work step. As a result, the electrode 10 is connected to the carrying device 2 in a cohesive manner in portions, and therefore in a permanent manner. The contact surface 10b here abuts on the carrying device 2 over the whole area.

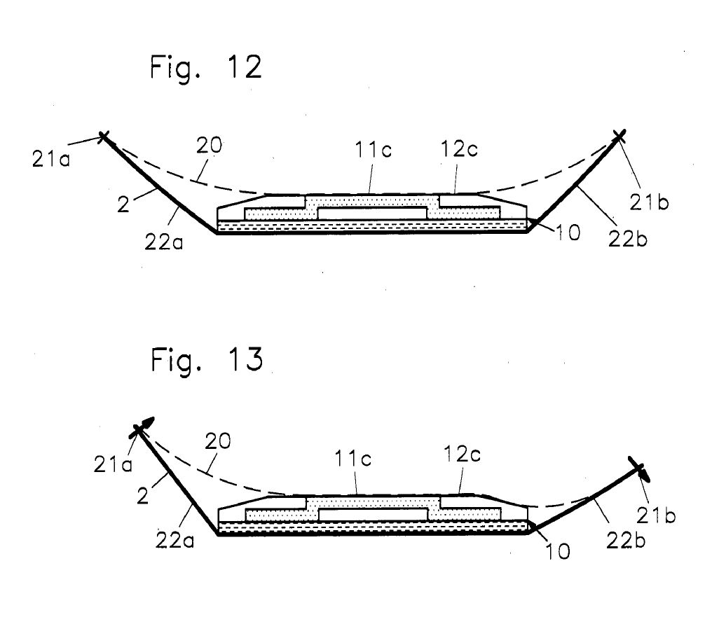

FIG. 10 shows alternative fastening of the electrode 10 on the carrying device 2. On the laterally formed limbs 11a thereof, the first component 11 includes means for fastening to a connection point 15. To this end, the edge portions 12a of the second component 12 have a narrower embodiment such that a protrusion of the edge portion 11b on the edge remains clear. This protrusion is surrounded by an edge 2c of the carrying device 2 and connected to the edge 2a at the connection point 15 by sewing, adhesively bonding or by other types of connection means. Here, the carrying device 2 engages around the edge portions 11b of the first component 11 and thereby provides additional protection. The cabling is housed in the interspace formed between the material of the carrying device 2 and the lower side of the electrode 10.

FIG. 11 depicts a development of the embodiment in FIG. 10, wherein the first component 11 is adhesively bonded to the thermoplastic connection ply 19. The second component 12 surrounds the first component 11 at the edge and engages around the edge portions 11b. In order to hold the first component 11 on the connection ply 19, the second component 12 is fused, i.e., cohesively connected, to the connection ply 19 at the edge. The edges 2a of the carrying device 2 engage around the edge portions 12a of the second component 12 and are cohesively connectable therewith by fusing or adhesively bonding as a result thereof.

FIGS. 12 and 13 show an electrode 10 attached to the carrying device 2, wherein the carrying device 2 is worn by a user. Here, with the electrically active surface thereof, the electrode 10 contacts a skin portion 20 of the user. In FIG. 12, the user is at rest and does not move. The skin portion 20 has two contact points 21a, 21b with the carrying device 2 and has a convex shape therebetween, wherein the electrode 10, in particular the skin-contact surfaces 11c, 12c of the first component 11 and of the second component 12, rest on the skin portion 20 in a planar manner. Portions 22a, 22b of the carrying device 2 extend between the contact points 21a, 21b. If the user moves, the carrying device 2 is also moved along, wherein the electrode 10 remains at its location as a result of the adhering second component 12. As a result of the electrode 10 being rigid compared to the carrying device 2, the portions 22a, 22b at the edge 12a of the electrode 10 are lifted off the body, as a result of which the contact pressure in the region of the active electrode surface, i.e., of the first component 11, increases. The carrying device 2 can therefore overall be worn with less contact pressure, as a result of which there is an improvement in the comfort of wear.

The portions 22a, 22b are shortened or lengthened during movement; in this case portion 22a is shortened and portion 22b is lengthened. The skin portion 20 abutting on the second component 12 is likewise moved, wherein there is a slight lift off on the side facing the portion 22a and an increased contact pressure on the skin portion 20 is generated on the other side facing the portion 22b. The first component 11 is not, however, subjected to any of the movements in this case. The skin portion 20 contacting there remains in its position. As a result, movement artifacts are reduced, if not even completely prevented.

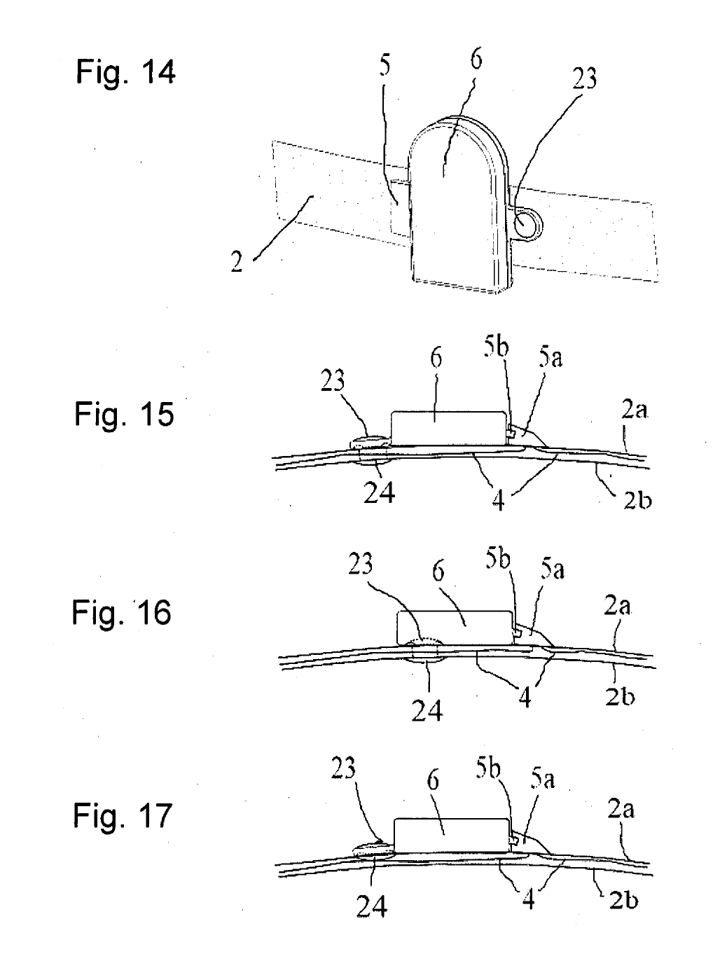

FIG. 14 depicts a perspective view of the measuring device 1 according to the present invention. On the carrying device 2, the data processing unit 6 is connected electrically thereto by means of the plug 5 and connected mechanically to the carrying device 2 by means of a connection apparatus 23, wherein the connection apparatus 23 is adhesively bonded, crimped or thermally fixed into the data processing unit.

FIG. 15 shows a side view of the measuring device 1 in a partial section. The plug 5 is made of a plug-in part 5a of the data processing unit 6 and a socket part 5b, wherein the latter part is fixed in the carrying device 2. The connection apparatus 23 is embodied as a snap fastener 23, which is attached to a counterpiece 24, which is arranged on the side 2b, facing away from the body, of the carrying device 2 and securely connected to same. The snap fastener 23 is arranged at the edge of the data processing unit 6 on a side lying opposite to the plug 5. This connection can additionally also be embodied as an additional, second electrical contact. The supply lines 4 are routed loosely within the carrying device 2, wherein they are routed around the connection point of the snap fastener 23 with the counterpiece 24 thereof.

In FIG. 16, the snap fastener 23 is attached not next to the data processing unit 6 but therebelow in an edge region. This reduces the overall spatial requirements of the data processing unit 6.

FIG. 17 depicts a variant of FIG. 15, wherein the counterpiece 24 is arranged within the carrying device 2 at the lower side of the body-facing side 2a.

The present invention is not limited to embodiments described herein; reference should be had to the appended claims.

### LIST OF REFERENCE SIGNS

1 Measuring device
2 Carrying device
2a Side of the carrying device facing away from the body
2b Side of the carrying device facing the body
2c Edge
3 Sensor
4 Electrical line/Cable
5 Plug
5a Plug-in part
5b Socket part
6 Data processing unit
7 Closure
10 Electrode
10a Component assemblage
10b Contact surface
11 First component
11a Limb
11b Edge portion
11c Contact surface of the first component
12 Second component
12a Edge portion
12c Contact surface of the second component
13 Electrical supply line
14a Conductive electrode surface
14b Substrate
15 Connection point/Opening
15a Perforation
16 Upper ply
17 Lower ply
18 Conductive textile
19 Connection ply
20 Skin portion of a user
21a, b Contact points
22a, b Portions of the carrying device
23 Connection apparatus/Snap fastener
24 Counterpiece

What is claimed is:

1-15. (canceled)

16. A skin-contact electrode for registering a biomedical vital parameter, the skin-contact electrode comprising:
a component assemblage comprising,
an outer edge,
a first component with a planer design, the first component comprising an electrically conductive material having a first component rigidity, and
a second component comprising an electrically insulating material having a second rigidity,
wherein,
the first component rigidity is greater than the second component rigidity, and
the outer edge is only formed by the second component.

17. The skin-contact electrode as recited in claim 16, wherein the component assemblage further comprises a component assemblage rigidity, and further comprising a carrier device comprising a carrier device rigidity, wherein the skin-contact electrode is configured to be fastened onto the carrier device, and the component assemblage rigidity is greater than the carrier device rigidity.

18. The skin-contact electrode as recited in claim 16, wherein the second component further comprises a flexible material and a static friction coefficient for an adhesion of the component assemblage to a skin surface.

19. The skin-contact electrode as recited in claim 18, wherein the flexible material is a thermoplastic polyurethane or a silicone.

20. The skin-contact electrode as recited in claim 16, wherein the first component further comprises a metal.

21. The skin-contact electrode as recited in claim 20, wherein the metal is at least one of a stainless steel, a film configured to be flexible and conductive comprising a film rigidity, and a textile configured to be conductive comprising a textile rigidity.

22. The skin-contact electrode as recited in claim 21, further comprising a contact surface, and a substrate comprising a substrate rigidity, wherein the film or the textile is connected at the contact surface to the substrate, and the substrate rigidity is greater than either of the textile rigidity and the film rigidity.

23. The skin-contact electrode as recited in claim 22, further comprising an electrical supply line, wherein the first component further comprises a first ply, a second ply, and a conductive textile which is pressed or adhesively bonded between the first ply and the second ply and which is electrically connected to the electrical supply line.

24. The skin-contact electrode as recited in claim 16, wherein the first component further comprises a first component skin-contact surface and the second component further comprises a second component skin-contact surface each of which are configured to lie in a plane.

25. The skin-contact electrode as recited in claim 16, wherein the first component further comprises at least one of a first component edge portion and a first component limb configured to be laterally formed, and the second component is configured to wholly or partly engage around at least one of the first component edge portion and the first component limb.

26. A measuring device for registering a biomedical vital parameter, the measuring device comprising:
a data processing unit; and
a carrying device comprising an elastic material and at least one skin-contact electrode as recited in claim 16,
wherein,
the carrying device is configured to be detachably connected to the data processing unit, and
the at least one skin-contact electrode is configured to be electrically connectable to the data processing unit.

27. The measuring device as recited in claim 26, wherein at least two skin-contact electrodes are attached to the carrying device, and wherein at least one of the at least two skin-contact electrodes is arranged on a section of the carrying device so that it is arrangeable on a user's back.

28. The measuring device as recited in claim 26, wherein the at least one skin-contact electrode is configured to be wholly or partly fused to the carrying device via a thermoplastic connection ply.

29. The measuring device as recited in claim 26, wherein,
the carrying device further comprises a carrying device surface,
the at least one skin contact electrode comprises a skin contact electrode contact surface,
the first component comprises a first component skin-contact surface, and
the skin contact electrode contact surface is fastened to the carrying device in a raised manner so that the first component skin-contact surface is level with, or raised in relation to, the carrying device surface.

30. The measuring device as recited in claim 26, wherein the carrying device further comprises a woven fabric, a loop-drawingly knitted fabric, or a loop-formingly knitted fabric, each respectively being made of a synthetic material.

31. The measuring device as recited in claim 26, wherein the carrying device is at least one of a chest strap, an armband, a bracelet, a foot band and a piece of clothing, and the carrying device is configured to wholly or partly engage around a user's body,

32. The measuring device as recited in claim 26, further comprising cables configured to be individually routed, wherein the carrying device further comprises at least one connection device configured as a plug and to establish at least one of an electrical connection and a mechanical contact, the at least one connection device being electrically connected to the at least one skin-contact electrode via the cables.
